(19) United States
(12) Patent Application Publication
(10) Pub. No.: US 2024/0216559 A1
(43) Pub. Date: Jul. 4, 2024

(54) ULTRAVIOLET LIGHT IRRADIATION SYSTEM AND ULTRAVIOLET LIGHT IRRADIATION METHOD

(71) Applicant: NIPPON TELEGRAPH AND TELEPHONE CORPORATION, Tokyo (JP)

(72) Inventors: Tomohiro TANIGUCHI, Musashino-shi, Tokyo (JP); Satoshi NARIKAWA, Musashino-shi, Tokyo (JP); Ayako IWAKI, Musashino-shi, Tokyo (JP); Takahito KIRIHARA, Musashino-shi, Tokyo (JP); Kazuhide NAKAJIMA, Musashino-shi, Tokyo (JP); Takashi MATSUI, Musashino-shi, Tokyo (JP); Nobutomo HANZAWA, Musashino-shi, Tokyo (JP); Yuto SAGAE, Musashino-shi, Tokyo (JP); Chisato FUKAI, Musashino-shi, Tokyo (JP)

(73) Assignee: NIPPON TELEGRAPH AND TELEPHONE CORPORATION, Tokyo (JP)

(21) Appl. No.: 18/562,769

(22) PCT Filed: Jun. 4, 2021
(86) PCT No.: PCT/JP2021/021429

§ 371 Date: Nov. 20, 2023

(51) Int. Cl. A61L 2/10; A61L 2/26; A61L 9/20

(52) U.S. Cl. CPC A61L 2/10; A61L 2/26; A61L 9/20; A61L 2202/11

(57) ABSTRACT

An object of the present invention is to provide an ultraviolet light irradiation system and an ultraviolet light irradiation method having a P-MP configuration capable of obtaining a predetermined effect such as sterilization.

An ultraviolet light irradiation system 301 according to the present invention includes an ultraviolet light source unit 11 that generates ultraviolet light, N (N is a natural number) irradiation units 13 that irradiate a desired site (irradiation target region ste) with the ultraviolet light, an optical switch 12 that switches the ultraviolet light to each of paths 14 to the irradiation units 13, and a switching control unit 15 that controls a switching operation of the optical switch 12 so as to give an opportunity to be supplied with an integrated light amount per unit time equal for each of the paths 14 on the basis of a transmission loss of the ultraviolet light for each of the paths 14 and an irradiation area where the irradiation unit 13 irradiates the ultraviolet light.

### TECHNICAL FIELD

The present disclosure relates to an ultraviolet light irradiation system that performs sterilization and virus inactivation using ultraviolet light.

### BACKGROUND ART

For the purpose of preventing infectious diseases and the like, there is an increasing demand for a system that performs sterilization and virus inactivation using ultraviolet light. There are roughly three categories of products of the system. Note that, in the present specification, the term “sterilization or the like” means sterilization and virus inactivation.

### (1) Mobile Sterilizing Robot

The product of Non Patent Literature 1 is an autonomous mobile robot that emits ultraviolet light. The robot can automatically perform sterilization or the like in a wide range without human intervention by emitting ultraviolet light while moving in a room in a building such as a hospital room.

### (2) Stationary Air Purifier

The product of Non Patent Literature 2 is a device that is installed on a ceiling or at a predetermined place in a room and performs sterilization or the like of air in the room while circulating the air. The device does not directly emit ultraviolet light and does not affect the human body, and thus highly safe sterilization can be performed.

### (3) Portable Sterilization Device

The product of Non Patent Literature 3 is a portable device in which an ultraviolet light source is mounted. A user can bring the device to a desired area and emit ultraviolet light. Thus, the device can be used in various places.

### CITATION LIST

### Non Patent Literature

Non Patent Literature 1: Website of Kantum Ushikata Co., Ltd.
(https://www.kantum.co.jp/product/sakkin_robot/sakkinn robot/UVD_robot), retrieved on Jun. 22, 2020
Non Patent Literature 2: Website of Iwasaki Electric Co., Ltd. (https://www.iwasaki.co.jp/optics/sterilization/air/air03.html), retrieved on Jun. 22, 2020
Non Patent Literature 3: Website of Funakoshi Co., Ltd. (https://www.funakoshi.co.jp/contents/68182), retrieved on Jun. 22, 2020

### SUMMARY OF INVENTION

### Technical Problem

However, the devices described in this non patent literature have the following problems.

### (A) Economic Efficiency

Since the product of Non Patent Literature 1 emits high-output ultraviolet light, the device becomes large-scale and expensive. Thus, the product of Non Patent Literature 1 has a problem that it is difficult to achieve an economical system.

### (B) Versatility

In the product of Non Patent Literature 1, since the ultraviolet light irradiation site is limited to a place where the robot can move and enter, it is difficult to irradiate a small place, a deep place, or the like with ultraviolet light.

In the product of Non Patent Literature 2, since the circulated indoor air is sterilized, a place to be sterilized or the like cannot be directly irradiated with ultraviolet light.

In the product of Non Patent Literature 3, for example, a thin pipe line or an area into which a person cannot enter cannot be irradiated with ultraviolet light.

As described above, the products of the non patent literatures have a problem with regard to the versatility of being able to irradiate any place with ultraviolet light.

### (C) Operability

The product of Non Patent Literature 3 is portable and can irradiate various places with ultraviolet light. However, in order to obtain a sufficient effect such as sterilization at the target site, the user is required to have skill and knowledge, and there is a problem in operability.

To solve these problems, an ultraviolet light irradiation system using an optical fiber as illustrated in FIG. 11 is considered. This ultraviolet light irradiation system transmits an ultraviolet ray from a light source using a thin and easily bendable optical fiber, and irradiates a place to be sterilized or the like with the ultraviolet rays output from the tip of the optical fiber in a pinpoint manner. Since any place can be irradiated with ultraviolet light only by moving the tip of the optical fiber, the problem (B) of versatility can be solved. In addition, since movement and setting of the ultraviolet light source are unnecessary and the user is not required to have skill and knowledge, the problem (C) of operability can also be solved. Furthermore, by employing a point to multipoint (P-MP) system configuration used in fiber to the home (FTTH), it is possible to perform sterilization of a plurality of sites or the like by sharing a single light source, and thus the problem (A) of economic efficiency can also be solved.

However, implementation of the P-MP configuration as an ultraviolet light irradiation system has the following problems.

### (1) Increase in Excess Loss

In the case of the P-MP configuration using the beam splitter as illustrated in FIG. 11, a multistage configuration is formed as the number of branches increases, excessive loss generated for each branch is accumulated, and there is a problem that it is difficult to obtain sufficient ultraviolet power from the tip of the optical fiber.

### (2) Switching Control

A P-MP configuration that solves the above problem of excessive loss is a P-MP configuration using an optical switch. If the optical switch is configured to communicate with any of the plurality of output ports by switching the path of the ultraviolet rays input from one port by a mechanical mechanism (move optical fiber, mirror, prism, and the like) or a mechanism by MEMS, the multistage branched configuration is not formed. Thus, the passage loss of the optical switch is hardly affected by the number of branches, and the problem of excessive loss can be solved.

On the other hand, in the configuration of the optical switch, since the ultraviolet rays are intermittently transmitted to each output port by the path switching operation, it is difficult to perform simultaneous and constant sterilization of a plurality of sites, or the like. Thus, in the ultraviolet light irradiation system having the P-MP configuration using the optical switch, it is necessary to perform switching control in consideration of an effect such as constant sterilization on a plurality of target sites.

However, the switching control method is not clarified.

In order to solve these problems, an object of the present invention is to provide an ultraviolet light irradiation system and an ultraviolet light irradiation method having a P-MP configuration capable of obtaining an effect such as predetermined sterilization.

### Solution to Problem

In order to achieve the above object, in an ultraviolet light irradiation system according to the present invention, an optical switch is disposed between an ultraviolet light source unit and an irradiation unit, and a path of ultraviolet light is switched at a predetermined timing.

Specifically, an ultraviolet light irradiation system according to the present invention includes:
an ultraviolet light source unit that generates ultraviolet light;
N (N is a natural number) irradiation units that irradiate a desired site with the ultraviolet light;
an optical switch that switches the ultraviolet light to each of paths to the irradiation units; and
a switching control unit that controls a switching operation of the optical switch so as to give an opportunity to be supplied with an integrated light amount per unit time equal for each of the paths on a basis of a transmission loss of the ultraviolet light for each of the paths and an irradiation area where the irradiation unit irradiates the ultraviolet light.

Further, an ultraviolet light irradiation method according to the present invention is an ultraviolet light irradiation method for irradiating a desired site with ultraviolet light generated by an ultraviolet light source unit from N (N is a natural number) irradiation units, the method including
giving, when the ultraviolet light is switched by an optical switch to each of paths to the irradiation units,
an opportunity to be supplied with an integrated light amount per unit time equal for each of the paths on a basis of a transmission loss of the ultraviolet light for each of the paths and an irradiation area where the irradiation unit irradiates the ultraviolet light.

The present ultraviolet light irradiation system has a system configuration in which the ultraviolet light source and the irradiation units are connected via the optical switch, and a path of ultraviolet light is switched at a predetermined timing by the optical switch. The timing is obtained from output power of the ultraviolet light source, the number of output ports of the optical switch, an optical fiber transmission loss to each target site, an ultraviolet light illuminance obtained at each target site, and necessary ultraviolet light energy. With this configuration, even in a configuration in which ultraviolet light is intermittently transmitted to each target site by the optical switch, it is possible to ensure that the time during which sterilization or the like is not performed at a plurality of target sites is suppressed to a certain value or less, and it is possible to reduce an infection risk.

Therefore, the present invention can provide an ultraviolet light irradiation system and an ultraviolet light irradiation method having a P-MP configuration capable of obtaining a predetermined effect such as sterilization.

The ultraviolet light irradiation system according to the present invention further includes
a sensor that detects presence of an avoiding object at which the ultraviolet light is to be avoided at the desired site, in which
when the sensor detects presence of the avoiding object at the desired site corresponding to one of the paths at the opportunity of the one path,
the switching control unit does not supply the ultraviolet light to the one path at the opportunity.

The present ultraviolet light irradiation system has a sensing function of detecting whether or not a person is present in the vicinity of each target site, and performs control to stop the ultraviolet light irradiation or shift the irradiation timing for the site where the person is detected.

For example, the switching control unit may separately give the opportunity to the one path after the avoiding object becomes absent, give the opportunity for the one path to another of the paths, or supply the ultraviolet light only at the opportunity after the avoiding object becomes absent.

Note that the inventions described above can be combined in any possible manner.

### Advantageous Effects of Invention

The present invention can provide an ultraviolet light irradiation system and an ultraviolet light irradiation method having a P-MP configuration capable of obtaining a predetermined effect such as sterilization.

### BRIEF DESCRIPTION OF DRAWINGS

FIG. 1 is a diagram illustrating an ultraviolet light irradiation system according to the present invention.

FIG. 2 is a diagram illustrating a cross-sectional structure of an optical fiber.

FIG. 3 is a diagram illustrating an intermittent distribution of ultraviolet light performed by an ultraviolet light irradiation system according to the present invention.

FIG. 4 is a diagram illustrating an ultraviolet light irradiation system according to the present invention.

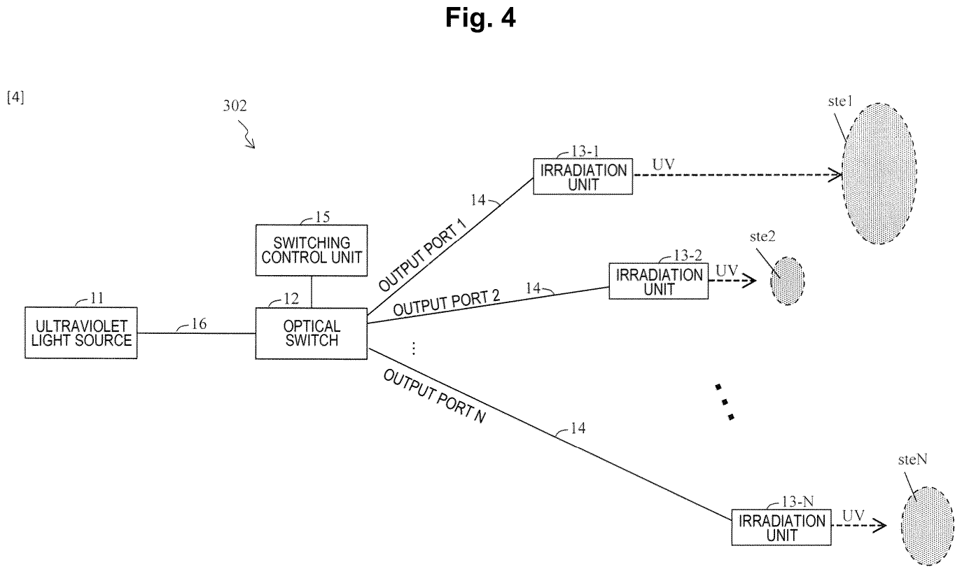

FIG. 5 is a diagram illustrating an intermittent distribution of the ultraviolet light performed by an ultraviolet light irradiation system according to the present invention.

FIG. 6 is a diagram illustrating an ultraviolet light irradiation system according to the present invention.

FIG. 7 is a diagram illustrating an intermittent distribution of the ultraviolet light performed by the ultraviolet light irradiation system according to the present invention.

FIG. 8 is a diagram illustrating an ultraviolet light irradiation method according to the present invention.

FIG. 9 is a diagram illustrating an intermittent distribution of the ultraviolet light performed by the ultraviolet light irradiation system according to the present invention.

FIG. 10 is a diagram illustrating an intermittent distribution of the ultraviolet light performed by the ultraviolet light irradiation system according to the present invention.

FIG. 11 is a diagram illustrating a P-MP configuration using a beam splitter.

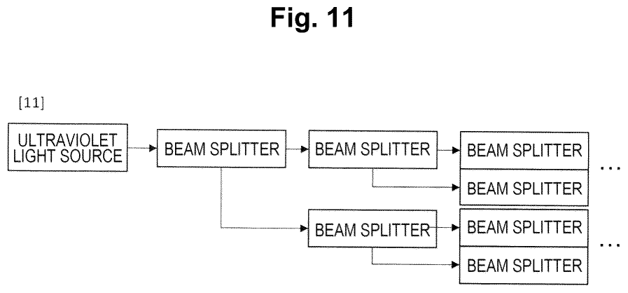

### DESCRIPTION OF EMBODIMENTS

Embodiments of the present invention will be described with reference to the accompanying drawings. The embodiment described below is an example of the present invention, and the present invention is not limited to the following embodiment. Note that like components are denoted by like reference numerals in this specification and the drawings.

### First Embodiment

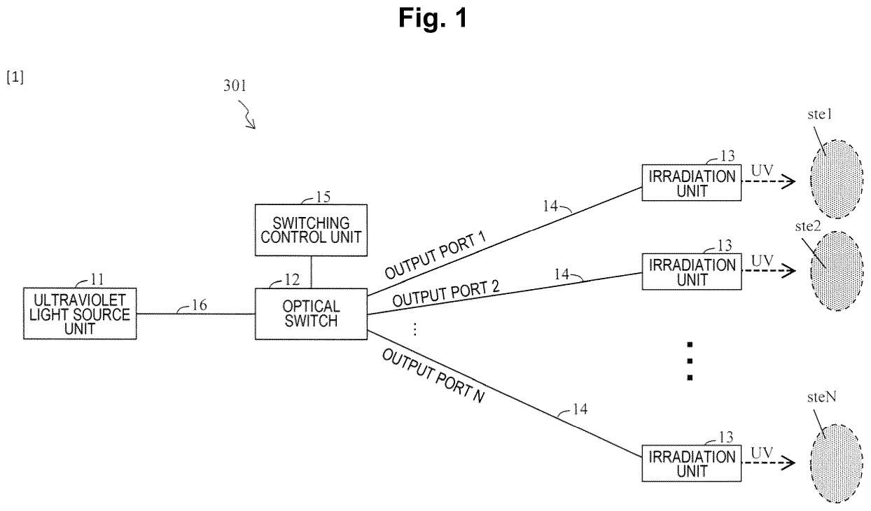

FIG. 1 is a diagram illustrating an ultraviolet light irradiation system 301 of the present embodiment. The present embodiment is a case where a transmission loss of ultraviolet light to an irradiation target region ste is equal to an irradiation area.

The ultraviolet light irradiation system 301 includes:
an ultraviolet light source unit 11 that generates ultraviolet light;
N (N is a natural number) irradiation units 13 that irradiate a desired site (irradiation target region ste) with the ultraviolet light;
an optical switch 12 that switches the ultraviolet light to each of paths 14 to the irradiation units 13; and
a switching control unit 15 that controls a switching operation of the optical switch 12 so as to give an opportunity to be supplied with an integrated light amount per unit time equal for each of the paths 14 on the basis of the transmission loss of the ultraviolet light for each of the paths 14 and the irradiation area where the irradiation unit 13 irradiates the ultraviolet light.

The ultraviolet light source unit 11 outputs light (ultraviolet light) in an ultraviolet region effective for sterilization and the like. The power of the ultraviolet light output from the ultraviolet light source unit 11 is defined as P [W]. The ultraviolet light source unit 11 and the optical switch 12 are connected by an optical fiber or a spatial path 16.

The switching control unit 15 controls the switching operation of the optical switch 12.

The optical switch 12 outputs the ultraviolet light from the ultraviolet light source unit 11 to any one path 14 of the plurality of output ports in accordance with an instruction from the switching control unit 15. Here, the time required for the optical switch 12 to switch the path is defined as T_{sw} [s]. The ultraviolet light output from the output ports 1 to N is emitted to the irradiation target regions 1 to N via the path 14 and the irradiation unit 13, respectively.

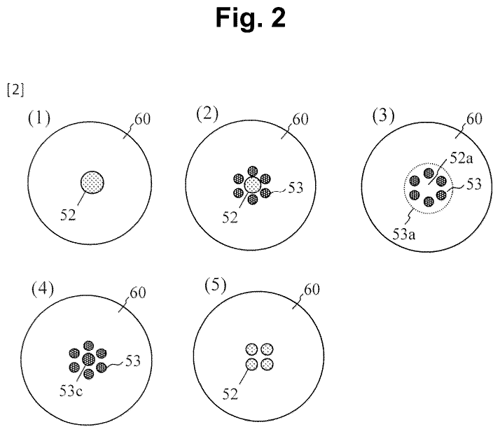

The path 14 propagates the ultraviolet light intermittently distributed by the optical switch 12 to each irradiation unit 13. The path 14 is an optical fiber. Since this is an optical fiber, the optical fiber can be laid in a small place where a robot or a device of the related art cannot enter. FIG. 2 is a diagram illustrating a cross section of an optical fiber usable for the path 14.

### (1) Solid Core Optical Fiber

This optical fiber has one solid core 52 having a refractive index higher than that of a clad 60, in the clad 60. As used herein, the term “solid” means “not hollow”. Furthermore, the solid core can also be made by forming an annular low refractive index region in the clad.

### (2) Hole-Assisted Optical Fiber

The optical fiber has the solid core 52 and a plurality of holes 53 arranged on the outer periphery thereof, in the clad 60. The medium of the hole 53 is air, and the refractive index of air is sufficiently smaller than that of quartz-based glass. Thus, the hole assisted optical fiber has a function of returning light leaked from the core 52 by bending or the like to the core 52 again, and has a small bending loss.

### (3) Hole Structure Optical Fiber

This optical fiber has a hole group 53a of the plurality of holes 53 in the clad 60, and has a refractive index effectively lower than that of a host material (glass or the like). This structure is called a photonic crystal fiber. This structure can have a structure in which a high refractive index core having a changed refractive index does not exist, and light can be confined using a region 52a surrounded by the holes 53 as an effective core region. Compared with an optical fiber having a solid core, the photonic crystal fiber can reduce the influence of absorption and scattering loss due to additives in the core, and can realize optical characteristics that cannot be realized by a solid optical fiber, such as reduction of bending loss and control of a nonlinear effect.

### (4) Hollow Core Optical Fiber

In the optical fiber, a core region is formed of air. When the clad region has a photonic bandgap structure formed by the plurality of holes or an anti-resonance structure formed of a thin glass wire, light can be confined in the core region. The optical fiber has a small nonlinear effect, and can supply a high-power or high-energy laser.

### (5) Coupling Core Type Optical Fiber

The optical fiber has a plurality of solid cores 52 that have a high refractive index arranged close to each other in the clad 60. The optical fiber guides light between the solid cores 52 by optical wave coupling. Since the coupling core type optical fiber can disperse and transmit light by the number of cores, the power can be increased accordingly and efficient sterilization can be performed, and then the coupling core type optical fiber has an advantage that fiber degradation due to ultraviolet rays can be alleviated and the life can be extended.

The irradiation unit 13 irradiates a predetermined target site (irradiation target region ste) to be sterilized or the like with the ultraviolet light transmitted through the path 14. The irradiation unit 13 includes an optical system such as a lens designed for a wavelength of ultraviolet light. Here, the loss from the ultraviolet light source unit 11 to the irradiation unit 13 (including the passage loss of the optical switch 12) is defined as L_{fiber} [a.u.], and the loss from the irradiation unit 13 to the irradiation target region ste is defined as Lₐᵢᵣ [a.u.]. Further, the area of the spot of the ultraviolet light with which the irradiation target region ste is irradiated is defined as S [m²].

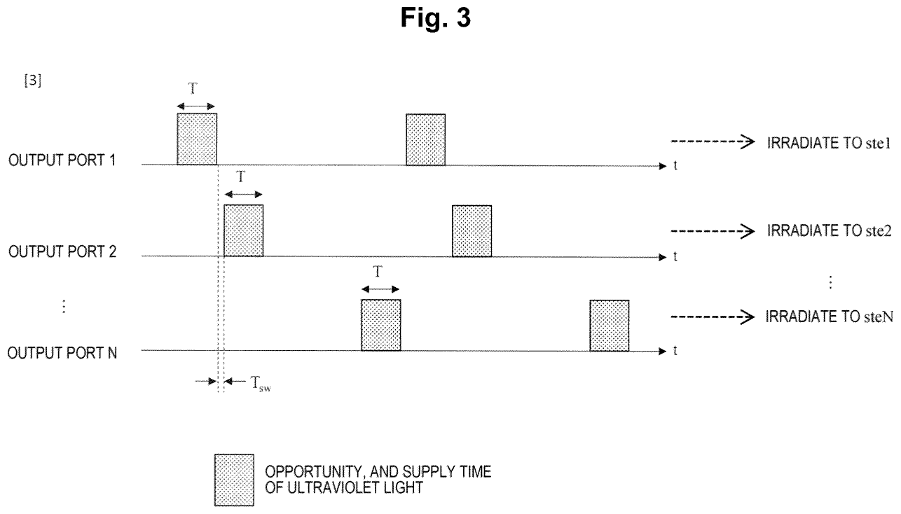

FIG. 3 is a diagram illustrating switching control of the optical switch 12 performed by the switching control unit 15. This control is an operation when the transmission loss of the ultraviolet light to the irradiation target region ste is equal to the irradiation area.

The switching control unit 15 controls the optical switch 12 to repeat an operation of giving an opportunity to supply the ultraviolet light for the time T to each path 14 (the output ports 1 to N of the optical switch). The time T is defined as a time obtained by Equation (1).

$$\begin{matrix}[ Math . 1 ] & \\ \frac{P}{{L}_{fiber} \cdot {L}_{air} \cdot S} \cdot T = E & ( 1 )\end{matrix}$$

Here, E [W·s/m²] is the amount of ultraviolet light necessary for sterilization and the like.

By operating the optical switch 12 in this way and supplying the ultraviolet light at the opportunity, the ultraviolet ray amount E [W·s/m²] necessary for sterilization and the like can be irradiated to all the irradiation target regions ste in the shortest cycle. That is, the ultraviolet light irradiation system 301 can ensure an effect such as sterilization for each irradiation target region ste at every time T_{inact} [s] of Expression (2).

$$\begin{matrix}[ Math . 2 ] & \\ {T}_{inact} = N \cdot ( T + {T}_{sw} ) & ( 2 )\end{matrix}$$

Note that, even if the switching control unit 15 controls the optical switch 12 so that the time of opportunity to supply ultraviolet light to each output port is T/M [s] (M is a natural number of 2 or more), a similar effect can be obtained.

### Second Embodiment

FIG. 3 is a diagram illustrating an ultraviolet light irradiation system 302 of the present embodiment. The present embodiment is a case where the transmission loss of ultraviolet light to the irradiation target region ste and the irradiation area are different. An ultraviolet light irradiation system 302 has the same structure as the ultraviolet light irradiation system 301 in FIG. 1, but the path length and the irradiation area S for each irradiation target region ste are different for each path 14. In the present embodiment, only portions different from the ultraviolet light irradiation system 301 will be described.

The power of the ultraviolet light output from the ultraviolet light source unit 11 is defined as P [W]. A time required for path switching by the optical switch 12 is defined as T_{sw} [s]. The loss from the ultraviolet light source unit 11 to the irradiation unit 13 (including the passage loss of the optical switch 12) is L_{fiber-1} [a.u.] for each path 14, and L_{fiber-2} [a.u.], . . . , L_{fiber-N} [a.u.] is defined. The loss from the irradiation unit 13 to each irradiation target region ste is defined as Lₐᵢᵣ₋₁ [a.u.], Lₐᵢᵣ₋₂ [a.u.], . . . , and L_{air-N} [a.u.]. Further, the area of the spot of the ultraviolet light irradiated to each irradiation target region ste is defined as S₁ [m²], S₂ [m²], . . . , S_{N} [m²].

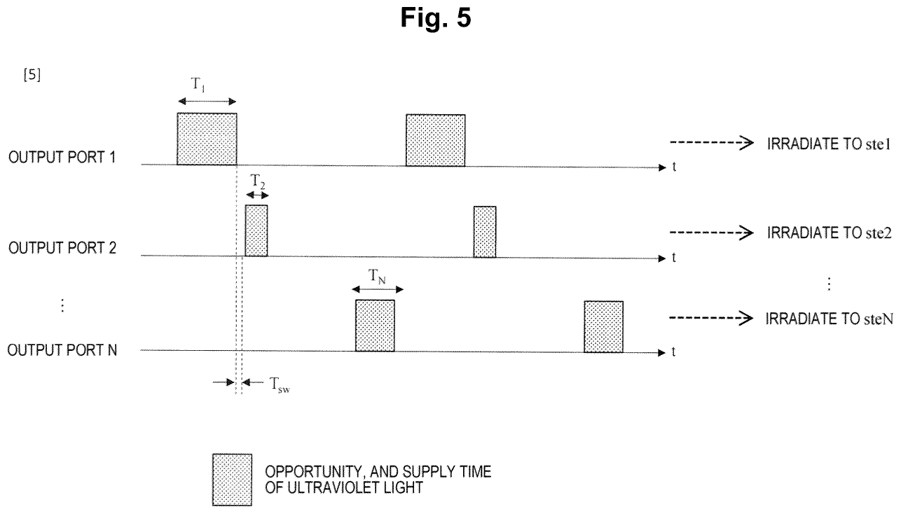

FIG. 5 is a diagram illustrating switching control of the optical switch 12 performed by the switching control unit 15. This control is an operation in a case where the transmission loss of the ultraviolet light to the irradiation target region ste and the irradiation area are different.

The switching control unit 15 controls the optical switch 12 to repeat an operation of giving an opportunity to supply the ultraviolet light for the time Ti to each of the output ports i (i=1, 2, . . . , N). Ti is defined as a time obtained by Expression (3).

$$\begin{matrix}[ Math . 3 ] & \\ \frac{P}{{L}_{fiber - i} \cdot {L}_{air - i} \cdot {S}_{i}} \cdot {T}_{i} = E & ( 3 )\end{matrix}$$

Here, E [W·s/m²] is the amount of ultraviolet light necessary for sterilization and the like.

By operating the optical switch 12 in this way and supplying the ultraviolet light at the opportunity, the ultraviolet ray amount E [W·s/m²] necessary for sterilization and the like can be irradiated to all the irradiation target regions ste in the shortest cycle. That is, the ultraviolet light irradiation system 302 can ensure an effect such as sterilization for each irradiation target region ste at every time T_{inact} [s] of Expression (4).

$$\begin{matrix}[ Math . 4 ] & \\ {T}_{inact} = {\sum }_{i = 1}^{N} {T}_{i} + N \cdot {T}_{sw} & ( 4 )\end{matrix}$$

Note that, even if the switching control unit 15 controls the optical switch 12 so that the time of opportunity to supply ultraviolet light to each output port is T/M [s] (M is a natural number of 2 or more), a similar effect can be obtained.

### Third Embodiment

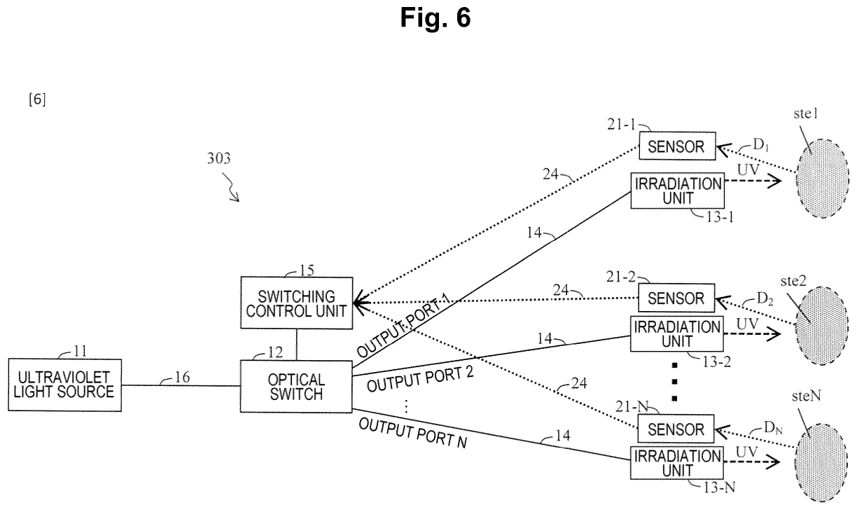

FIG. 6 is a diagram illustrating an ultraviolet light irradiation system 303 of the present embodiment. The present embodiment is a case where each irradiation target region ste is further provided with a function of detecting presence or absence of an avoiding object (a person or an animal) at which the ultraviolet light is to be avoided.

That is, the ultraviolet light irradiation system 303 further includes the sensor 21 that detects the presence of the avoiding object at which the ultraviolet light is to be avoided at the desired site (irradiation target region ste) with respect to the ultraviolet light irradiation system 301 of FIG. 1 and the ultraviolet light irradiation system 302 of FIG. 2, and
when the sensor 21 detects the presence of the avoiding object at the desired site (irradiation target region ste) corresponding to one of the paths 14 at the opportunity of the one path 14, the switching control unit 15 does not supply the ultraviolet light to the one path 14 at the opportunity.

In the present embodiment, only portions different from the ultraviolet light irradiation system (301 or 302) will be described.

Each sensor 21-i acquires various types of information Dᵢ (i=1, 2, . . . , N) of each irradiation target region ste and detects the presence or absence of the avoiding object. As a combination of the “sensor” and the “various types of information”, for example, the following is conceivable.

When the sensor 21 is a camera, the various types of information Dᵢ are video information.

When the sensor 21 is an infrared sensor, the various types of information Dᵢ are temperature information.

When the sensor 21 is a microphone, the various types of information Dᵢ are voice information.

Each sensor 21-i transmits information Dᵢ to the switching control unit 15 via the path 24. A wired communication method (for example, a wired LAN or the like) or a wireless communication method (for example, a wireless LAN or the like) can be applied to the path 24.

The switching control unit 15 performs switching control of the optical switch 12 on the basis of the information Dᵢ.

### Example 1

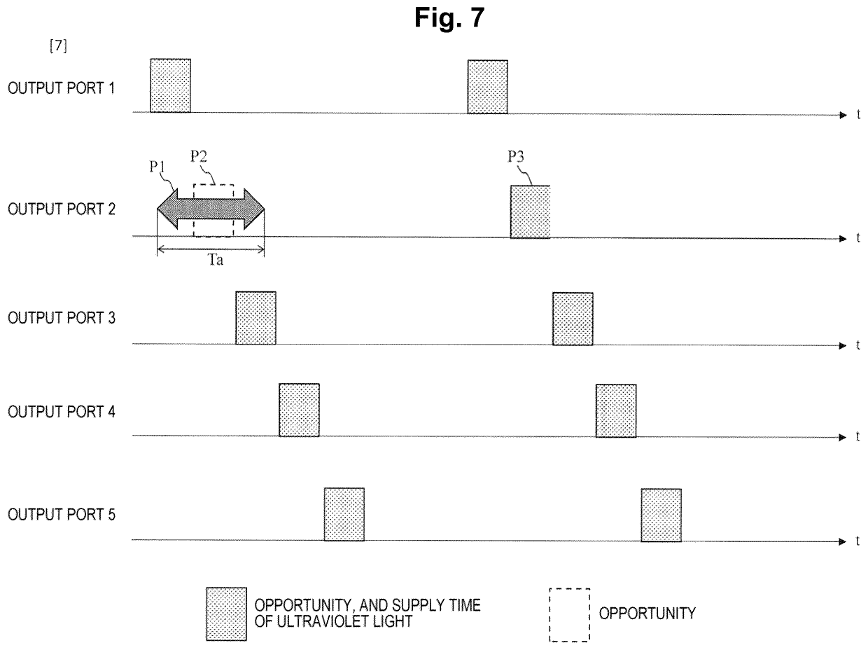

FIG. 7 is a diagram illustrating switching control of the optical switch 12 performed by the switching control unit 15.

The switching control unit 15 adds the following control to the switching control described with reference to FIGS. 3 and 5. The control to be added is to stop the supply of the ultraviolet light to the output port to the irradiation target region ste when the avoiding object is detected in the irradiation target region ste, and to perform the supply of the ultraviolet light to the output port to the irradiation target region ste when the avoiding object is not detected in the irradiation target region ste at the next opportunity.

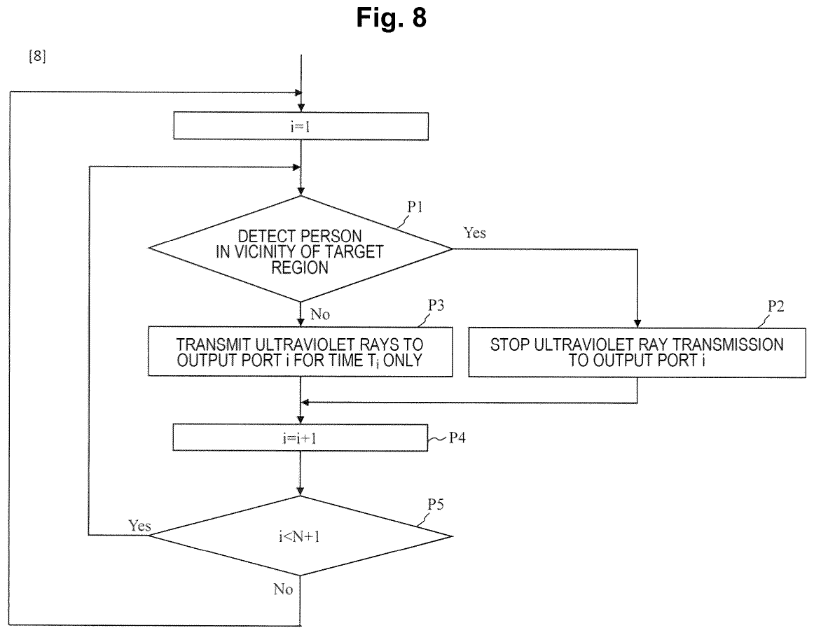

FIG. 8 is a flowchart illustrating the control.

Specifically described with reference to the examples of FIGS. 6 and 7,
Step P1: the sensor 21-2 detects that a person is present in the vicinity of the irradiation target region ste2 for a certain period Ta,
Step P2: the switching control unit 15 stops the supply of the ultraviolet light to the output port 2 (stops the ultraviolet light to the irradiation target region ste2) at an opportunity included in the period Ta on the basis of the information D2 from the sensor 21-2,
Step P3: when the sensor 21-2 does not detect a person at the next opportunity, the switching control unit 15 performs supply of the ultraviolet light to the output port 2 at the next opportunity,
Step P4: the presence or absence of a person for the next irradiation target region ste-i (ste4) is determined, and
Step P5: after confirming up to the last irradiation target region ste-N, the presence or absence of a person is determined from the irradiation target region ste-1 as the next opportunity.
and.

Although the ultraviolet light is harmful to the human body depending on the wavelength, the ultraviolet light irradiation system 303 can perform the control described in the control modes 1 and 2 while avoiding the ultraviolet light irradiation to a person by this control.

### Example 2

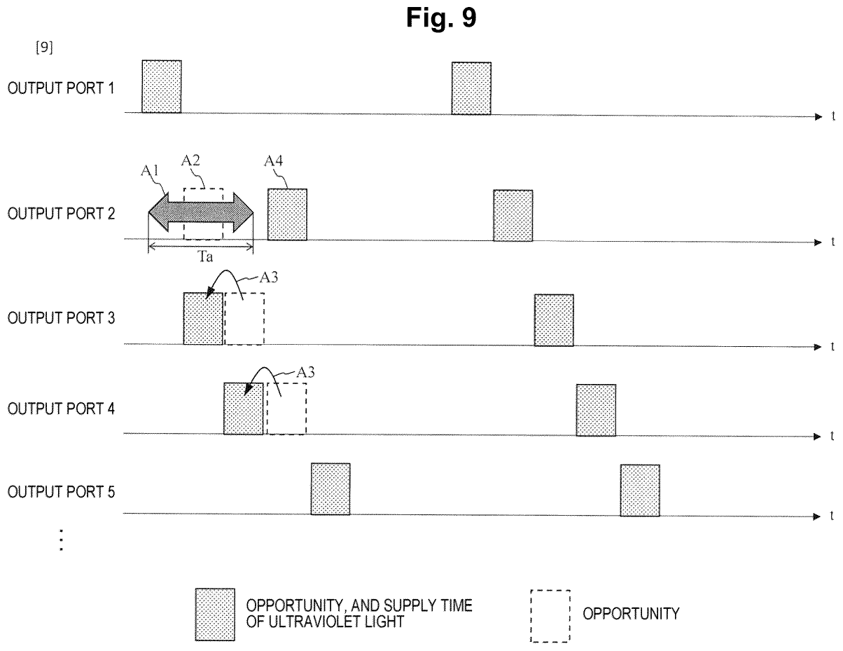

FIG. 9 is a diagram illustrating switching control of the optical switch 12 performed by the switching control unit 15.

The switching control unit 15 adds the following control to the switching control described with reference to FIGS. 3 and 5. The control to be added is to stop the supply of the ultraviolet light to the output port to the irradiation target region ste when the avoiding object is detected in the irradiation target region ste, to supply the ultraviolet light by shortening the opportunity of another output port while the avoiding object is detected in the irradiation target region ste, and to supply the ultraviolet light by giving the opportunity to the output port after the avoiding object can no longer be detected in the irradiation target region ste.

Specific description will be given with reference to the examples of FIGS. 6 and 9.
Step A1: the sensor 21-2 detects that a person is present in the vicinity of the irradiation target region ste2 for the certain period Ta,
Step A2: the switching control unit 15 stops the supply of the ultraviolet light to the output port 2 (stops the ultraviolet light to the irradiation target region ste2) at an opportunity included in the period Ta on the basis of the information D2 from the sensor 21-2,
Step A3: the switching control unit 15 checks whether or not there is a person in the other irradiation target region ste for the certain period Ta, and when there is no person in the other irradiation target region ste, sequentially advances opportunities of the other output ports (the output ports 3 and 4 in the drawing) to supply the ultraviolet light to the output ports 3 and 4, and
Step A4: after the sensor 21-2 no longer detects a person, the switching control unit 15 gives an opportunity to the output port 2 to supply the ultraviolet light to the output port 2.

The ultraviolet light irradiation system 303 can also perform the control described in the control modes 1 and 2 while avoiding the ultraviolet irradiation of the person by this control, similarly to the control example of FIG. 7.

In addition, in the control example of FIG. 7, when a person is detected in the irradiation target region ste, it is necessary to wait for the supply of the ultraviolet light to the corresponding site until the next opportunity, and the time when the effect such as sterilization cannot be obtained becomes long. However, in this control, since the supply of the ultraviolet light is performed as soon as there is no person in the irradiation target region ste, the time when the effect such as sterilization cannot be obtained can be shortened as compared with the control of FIG. 7. As a consequence, the control example of FIG. 9 can reduce an infection risk.

### Example 3

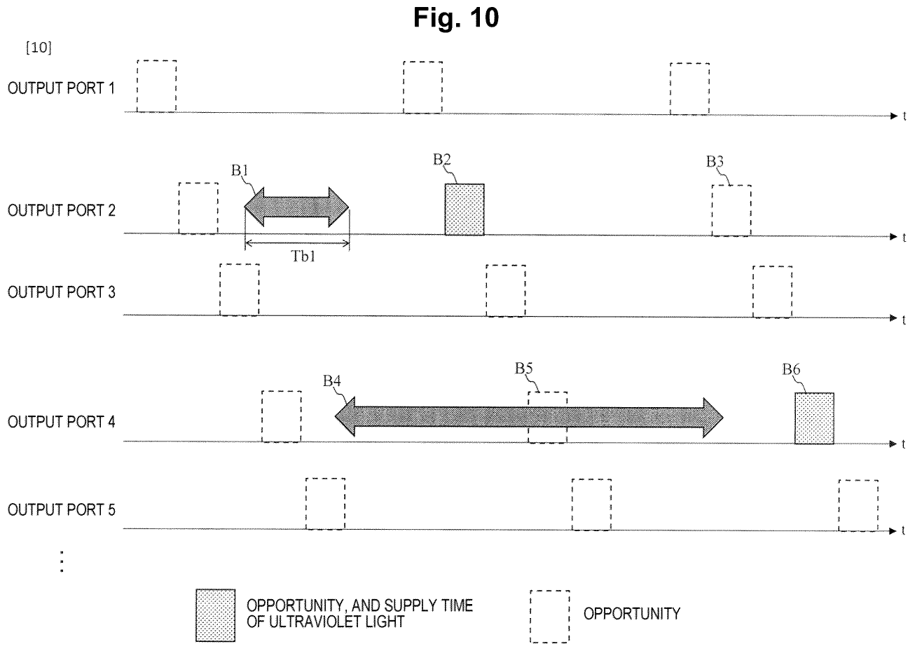

FIG. 10 is a diagram illustrating switching control of the optical switch 12 performed by the switching control unit 15.

The switching control unit 15 adds the following control to the switching control described with reference to FIGS. 3 and 5. The control to be added is such that, when an avoiding object is detected in the irradiation target region ste or when a touch of a person on an object present in the irradiation target region ste is detected, it is determined that sterilization or the like is necessary, and the ultraviolet light is supplied to the output port to the irradiation target region ste at the next opportunity when the avoiding object is not detected in the irradiation target region ste, and in other cases, the ultraviolet light is not supplied to the output port at each opportunity.

This will be specifically described with reference to the examples of FIGS. 6 and 10.
Step B1: the sensor 21-2 detects that a person has visited the irradiation target region ste2 or that a person has touched an object present in the irradiation target region ste2. Note that it is assumed that a certain period Tb1 is between opportunities given to the irradiation target region ste2.
Step B2: If a person is not detected at the next opportunity after the period Tb1 on the basis of the information D2 from the sensor 21-2, the switching control unit 15 supplies the ultraviolet light to the output port 2 at the next opportunity.
Step B3: the switching control unit 15 does not supply the ultraviolet light to the output port 2 at the current opportunity unless it is detected that a person has been present in the irradiation target region ste2 or a person has touched an object present in the irradiation target region ste2 from the previous opportunity to the current opportunity on the basis of the information D2 from the sensor 21-2.
Step B4: the sensor 21-4 detects that a person has visited the irradiation target region ste4 or that a person has touched an object present in the irradiation target region ste4. Note that a certain period Tb2 is defined as a period over one opportunity or a plurality of opportunities given to the irradiation target region ste4.
Step B5: on the basis of the information D4 from the sensor 21-4, the switching control unit 15 stops the supply of the ultraviolet light to the output port 4 at an opportunity included in the period Tb2 (stops the ultraviolet light to the irradiation target region ste4).
Step B6: if a person is not detected at the next opportunity after the period Tb2 on the basis of the information D4 from the sensor 21-4, the switching control unit 15 supplies ultraviolet light to the output port 4 at the next opportunity.

The ultraviolet light irradiation system 303 can also perform the control described in the control modes 1 and 2 while avoiding the ultraviolet irradiation of the person by this control, similarly to the control example of FIG. 7.

In this control, in addition to the operations of the control modes 1 and 2, it is possible to avoid ultraviolet irradiation to a person and to avoid unnecessary ultraviolet irradiation to a site where sterilization or the like is unnecessary.

### Advantageous Effects of Invention

The present invention has a characteristic in a system configuration in which an ultraviolet light source and an irradiation unit installed in the vicinity of a target site for sterilization or the like are connected via an optical switch, and a path in the optical switch is switched at a predetermined timing. According to this characteristic, it is possible to prevent a decrease in ultraviolet light power due to the multistage configuration for the irradiation target region, and it is possible to reduce the infection risk while securing a certain effect such as sterilization.

### REFERENCE SIGNS LIST

11 Ultraviolet light source unit
12 Optical switch
13, 13-1, . . . , 13-N Irradiation unit
14 Optical fiber
15 Switching control unit
16 Path
52 Solid core
52a Region
53 Hole
53a Hole group
53c Hole
60 Clad
301 to 303 Ultraviolet light irradiation system
ste1, ste2, . . . , steN Irradiation target region (region to be irradiated with ultraviolet light)

1. An ultraviolet light irradiation system, comprising:
an ultraviolet light source unit that generates ultraviolet light;
N (N is a natural number) irradiation units that irradiate a desired site with the ultraviolet light;
an optical switch that switches the ultraviolet light to each of paths to the irradiation units; and
a switching control unit that controls a switching operation of the optical switch so as to give an opportunity to be supplied with an integrated light amount per unit time equal for each of the paths on a basis of a transmission loss of the ultraviolet light for each of the paths and an irradiation area where the irradiation unit irradiates the ultraviolet light.

2. The ultraviolet light irradiation system according to claim 1, further comprising
a sensor that detects presence of an avoiding object at which the ultraviolet light is to be avoided at the desired site, wherein
when the sensor detects presence of the avoiding object at the desired site corresponding to one of the paths at the opportunity of the one path,
the switching control unit does not supply the ultraviolet light to the one path at the opportunity.

3. The ultraviolet light irradiation system according to claim 2, wherein the switching control unit separately gives the opportunity to the one path after the avoiding object becomes absent.

4. The ultraviolet light irradiation system according to claim 2, wherein the switching control unit gives the opportunity for the one path to another of the paths.

5. The ultraviolet light irradiation system according to claim 2, wherein the switching control unit supplies the ultraviolet light only at the opportunity after the avoiding object becomes absent.

6. An ultraviolet light irradiation method for irradiating a desired site with ultraviolet light generated by an ultraviolet light source unit from N (N is a natural number) irradiation units, the method comprising
giving, when the ultraviolet light is switched by an optical switch to each of paths to the irradiation units,
an opportunity to be supplied with an integrated light amount per unit time equal for each of the paths on a basis of a transmission loss of the ultraviolet light for each of the paths and an irradiation area where the irradiation unit irradiates the ultraviolet light.
